(19) United States
(12) Patent Application Publication
(10) Pub. No.: US 2020/0078409 A1
(43) Pub. Date: Mar. 12, 2020

(54) METHOD FOR TREATMENT OF INFERTILITY, PHARMACEUTICAL COMPOSITION FOR TREATMENT OF INFERTILITY AND METHOD FOR PRODUCING THE SAME

(71) Applicant: FUKUOKA UNIVERSITY, Fukuoka-shi (JP)

(72) Inventors: Shingo MIYAMOTO, Fukuoka-shi (JP); Fusanori YOTSUMOTO, Fukuoka-shi (JP)

(73) Assignee: FUKUOKA UNIVERSITY, Fukuoka-shi (JP)

(21) Appl. No.: 16/684,142

(22) Filed: Nov. 14, 2019

Related U.S. Application Data
(62) Division of application No. 15/819,654, filed on Nov. 21, 2017.

(30) Foreign Application Priority Data
Apr. 12, 2017 (JP) 2017-079148

(51) Int. Cl. A61K 35/28; A61K 47/36; A61K 9/00; A61P 15/08; A61K 35/44; A61K 31/728

(52) U.S. Cl. CPC A61K 35/28; A61K 47/36; A61K 9/0034; A61K 9/0039; A61K 35/44; A61K 31/728; A61P 15/08

(57) ABSTRACT

A pharmaceutical composition contains adipose tissue derived reproductive cells including adipose stem cells, vascular endothelial cells and vascular pericytes. The composition is administrated into an intrauterine cavity of a subject and used for treatment of infertility. A method for producing the pharmaceutical composition includes treating an adipose tissue with a disaggregation agent to obtain a disaggregated tissue; concentrating reproductive cells from the disaggregated tissue by centrifugal separation treatment; and recovering concentrated reproductive cells. A method for treatment of infertility of a subject includes administering a pharmaceutical composition into an intrauterine cavity of a subject. The pharmaceutical composition contains adipose tissue derived reproductive cells including adipose stem cells, vascular endothelial cells and vascular pericytes.

### CROSS-REFERENCE TO RELATED PATENT APPLICATION

This is a divisional application of U.S. patent application Ser. No. 15/819,654, filed Nov. 21, 2017, which claims benefit of Japanese Patent Application No. 2017-079148, filed on Apr. 12, 2017 the entire disclosure of which is incorporated herein by reference.

### BACKGROUND

### Field of the Invention

The present invention relates to a method for treatment of infertility, a pharmaceutical composition to be used for treatment of infertility and a method for producing the pharmaceutical composition.

### Description of the Related Art

In Japan, one in six pairs of married couples is currently diagnosed as infertile. Causes of infertility include uterine factors, cervical factors, ovulation factors and oviduct factors as female factors. For the three factors other than uterine factors, already established therapeutic methods such as artificial insemination and in vitro fertilization are available. The uterine factors include organic diseases (e.g. uterine fascia and uterine malformations) in which the structure of the uterus itself has a problem, and it is possible to cure these diseases by surgery. On the other hand, there is no effective therapeutic method for infertility caused by endometrial thinning, so-called implantation deficiency, i.e. a functional disease in which the uterine function has a problem.

Japanese Patent Laid-open Publication No. 2012-75439 describes an automated system for separating reproductive cells from living tissues, and describes that the reproductive cells are applicable to various diseases and disorders. In addition, Japanese Patent Laid-open Publication No. 2012-51923 describes that reproductive cells obtained from adipose tissues may be used for therapy of cardiovascular conditions. Further, Human Reproduction, 2012, Vol. 27, Suppl. 2, Abstract Number: P-587 describes that when adipose derived stem cells (ASC) obtained by cell culture were injected into a subendometrial region in an infertile female having an endometrium thickness of 3 to 5 mm in previous unsuccessful in vitro fertilization (IVF), the endometrium had a thickness of 7 mm and a thickness of 8 mm in a second artificial period and a third artificial period, respectively, after administration of ASC, and uterine pregnancy occurred on the 30th day after implantation of a blastocyst in the third period.

### SUMMARY

A pharmaceutical composition contains adipose tissue derived reproductive cells including adipose stem cells, vascular endothelial cells and vascular pericytes. The composition is administrated into an intrauterine cavity of a subject and used for treatment of infertility. A method for producing the pharmaceutical composition includes: treating an adipose tissue with a disaggregation agent to obtain a disaggregated tissue; concentrating reproductive cells from the disaggregated tissue by centrifugal separation treatment; and recovering concentrated reproductive cells. A method for treatment of infertility of a subject includes administering a pharmaceutical composition into an intrauterine cavity of a subject and the pharmaceutical composition contains adipose tissue derived reproductive cells including adipose stem cells, vascular endothelial cells and vascular pericytes.

### BRIEF DESCRIPTION OF THE DRAWINGS

FIG. 1A is an electron micrograph showing one example of an adipose tissue;

FIG. 1B is a schematic view showing one example of an adipose tissue;

FIG. 2A is a detection result of Sca-1 in flow cytometry of mouse ADRCs;

FIG. 2B is a detection result of CD105 in flow cytometry of mouse ADRCs;

FIG. 2C is a detection result of CD29 in flow cytometry of mouse ADRCs;

FIG. 2D is a detection result of CD45 in flow cytometry of mouse ADRCs;

FIG. 3 is a tissue staining view showing an endometrium of a normal mouse;

FIG. 4A is a tissue staining view showing a sagittal section of a uterus of a control group mouse;

FIG. 4B is a tissue staining view showing a transverse section of a uterus of a control group mouse;

FIG. 5A is a tissue staining view showing a sagittal section of a uterus of a test group mouse;

FIG. 5B is a tissue staining view showing a transverse section of a uterus of a test group mouse;

FIG. 6A is a view showing an endometrial proliferation capability of mouse ADRCs;

FIG. 6B is a view showing a capability of increasing the number of uterine glands by mouse ADRCs;

FIG. 7 is a view showing an endometrial proliferation capability of mouse ADRCs;

FIG. 8A is a view showing an endometrial proliferation capability of human ADRCs with respect to a mouse;

FIG. 8B is a view showing a capability of increasing the number of uterine glands by human ADRCs with respect to a mouse;

FIG. 9A is an immunofluorescence staining view showing a sagittal section of a uterus of a control group mouse;

FIG. 9B is an immunofluorescence staining view showing a sagittal section of a uterus of a test group mouse;

FIG. 10 is a view showing an angiogenesis capability of mouse ADRCs;

FIG. 11 is a schematic view of an experiment schedule used for effectiveness evaluation of ADRCs;

FIG. 12A is a view showing an isolated uterus after implantation of an embryo of a control group mouse;

FIG. 12B is a view showing an isolated uterus after implantation of an embryo of a test group mouse;

FIG. 13 is a view showing a result of evaluating an implantation ratio of mouse ADRCs; and

FIG. 14 is a view showing a result of evaluating an implantation ratio of human ADRCs with respect to a mouse.

### DETAILED DESCRIPTION

The present invention provides a pharmaceutical composition capable of ameliorating infertility and a method for producing the pharmaceutical composition.

Specific means for the above-mentioned problems are as follows, and the present invention encompasses the following aspects.

A first aspect is a pharmaceutical composition for intrauterine cavity administration which includes adipose tissue derived reproductive cells including adipose stem cells, vascular endothelial cells and vascular pericytes, and is used for treatment of infertility.

A second aspect is a method for producing a pharmaceutical composition for intrauterine cavity administration which is used for treatment of infertility. The method includes treating an adipose tissue with a disaggregation agent to obtain a disaggregated tissue; concentrating reproductive cells from the disaggregated tissue by centrifugal separation treatment; and recovering concentrated reproductive cells.

A third aspect is a method for treatment of infertility of a subject. The method includes administering a pharmaceutical composition to an intrauterine cavity of a treatment subject, the pharmaceutical composition containing adipose tissue derived reproductive cells including adipose stem cells, vascular endothelial cells and vascular pericytes.

In the present specification, the content of each component in a composition, when a plurality of substances corresponding to each component are present in the composition, means the total amount of the plurality of substances present in the composition unless otherwise specified. Hereinafter, the present invention will be described on the basis of embodiments. It should be noted that the embodiments described below are intended for embodying the technical idea of the present invention, and are illustrative of a pharmaceutical composition to be used for treatment of infertility and a method for producing the pharmaceutical composition. The present invention is not limited to a pharmaceutical composition and a method for producing the pharmaceutical composition as described below.

### Pharmaceutical Composition

The pharmaceutical composition to be used for treatment of infertility according to the present disclosure is characterized by including adipose tissue derived reproductive cells (hereinafter, also referred to as “ADRCs”). As described in Cytotherapy (2008), Vol. 10, No. 4, 417-426, Japanese Journal of Transfusion and Cell Therapy, Vol. 59, No. 3 59 (3): 450-456, 2013, ADRCs include vascular endothelial cells and vascular pericytes in addition to adipose cell derived stem cells. It is considered that ADRCs have a property of promoting angiogenesis and tissue repair for example, and are capable of repairing a thinned endometrium when administered to the intrauterine cavity so as to be in contact with the endometrium. It is considered that as a result, it is able to improve the implantation ratio of the fertilized egg to the endometrium, resulting in amelioration of infertility.

The pharmaceutical composition is used for treatment of infertility. The “treatment” used herein may be any treatment applied for infertility, and mention is made of, for example, therapy, amelioration and prevention of aggravation of infertility.

Among female factors of infertility, an implantation failure is known as a functional disease in which the uterine function has a problem as a uterine factor. The implantation failure refers to a state of being infecund or having a miscarriage although good embryos have been implanted three or more times by in vitro fertilization. One of the causes of the implantation failure is that the endometrium is not thickened (also referred to as endometrial thinning). The endometrial thinning is, for example, a state in which due to a decrease in secretion of female hormones (e.g. estrogen and progesterone), proliferation of the endometrium suitable for implantation does not occur. An attempt is made to ameliorate endometrial thinning by administering female hormone, but there may be no effect. While a cause thereof is unknown, the proliferation capability of the endometrium itself may be maintained.

The implantation failure resulting from endometrial thinning is considered as follows. After fertilization is established, a fertilized egg moves from an oviduct into a uterus, and is implanted in an endometrium to establish pregnancy. However, it is considered that if the endometrium is thinned, the fertilized egg moving from the oviduct into the uterus is unable to adhere to the endometrium, and thus unable to be successfully implanted in the endometrium. For example, it is known that a thinned endometrium is poor in angiogenesis, and has low reactivity to estrogen having an endometrial proliferation action. In this case, even when a hormonal drug therapy is applied for an implantation failure resulting from endometrial thinning, the therapy is not effective. Here, the present inventors have conceived a therapeutic method in which adipose stem cells are injected into a uterus for endometrial thinning, leading to completion of the present invention.

Adipose tissue derived reproductive cells (ADRCs) for use in the present disclosure are reproductive cells that may be prepared from an adipose tissue by a method as described later, include, for example, adipose stem cells and/or progenitor cells, and further include vascular endothelial cells and vascular pericytes. In particular, it is preferable that ADRCs include at least adipose stem cells, vascular endothelial cells and vascular pericytes. It is preferable that ADRCs have at least an angiogenesis inducing action (also referred to as an angiogenesis capability), and it is also preferable that ADRCs have an endometrial proliferation capability.

Preferably, ADRCs express at least one selected from the group consisting of Sca-1, CD 105 and CD 29 as a positive marker. More preferably, ADRCs express all of Sca-1, CD 105 and CD 29. In addition, it is preferable that ADRCs do not express CD 45 that is a negative marker. Details of surface antigens of ADRCs are described in Cytotherapy (2008), Vol. 10, No. 4, 417-426.

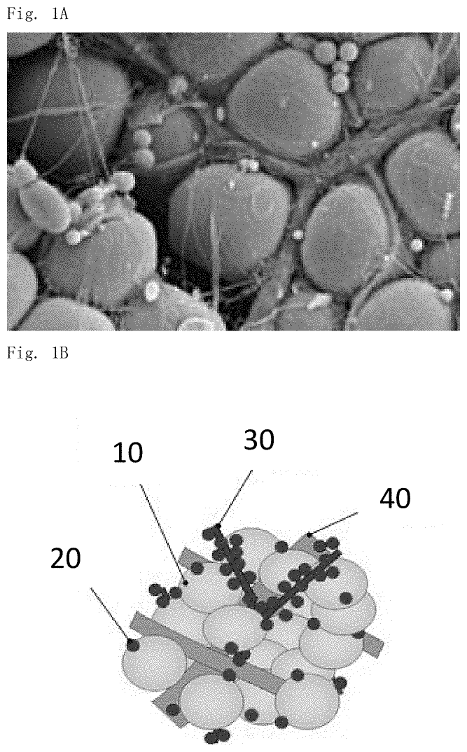

An adipose tissue from which reproductive cells are obtained may be present in any part of a living body, and is preferably a subcutaneous fat present in a subcutaneous tissue. The subcutaneous fat may be collected from any part of a living body, for example a waist-back part or a femur. One example of the subcutaneous fat is shown in a microscopic image in FIG. 1A (modified from FIG. 1A in Matsumoto D, et al., Tissue Engineering. December 2006, 12 (12): 3375-3382) and a schematic view in FIG. 1B. In FIG. 1B, adipose stem cells 20 as small cells are present around adipose cells 10 as large cells. In FIG. 1B, further a blood vessel 30 and an extracellular matrix 40 are shown. The abundance ratio of adipose cells 10 in a subcutaneous fat is, for example, about 20%, and the abundance ratio of adipose stem cells 20 is, for example, from about 1% to 5%. In addition, the blood vessel 30 is included at a rate of from about 15% to 20%. Adipose stem cells may differentiate into various organs such as a heart, a skeletal muscle, a bone/cartilage and a blood vessel. In addition, adipose stem cells secrete a physiologically active substance and have an action of inducing angiogenesis.

ADRCs contained in the pharmaceutical composition may be ADRCs prepared at the time of use or cryopreserved ADRCs. Cryopreserved ADRCs are more preferable because they can be administered day after day, and may be expected to improve the implantation ratio. For cryopreservation of ADRCs, cryopreservation conditions for commonly used cells may be applied. ADRCs may be prepared from an adipose tissue collected from an administration subject or, may be prepared from an adipose tissue collected from an individual different from the administration subject. Further, ADRCs may be prepared from an adipose tissue collected from an animal different in species from the administration subject.

The pharmaceutical composition may further contain other components such as pharmaceutically acceptable excipients as necessary in addition to ADRCs. Examples of other components may include surfactants, physiologically active substances, stabilizers and liquid media in addition to pharmaceutically acceptable excipients. Specific examples of other components may include glycosaminoglycans such as hyaluronic acid and chondroitin sulfate, and derivatives of the glycosaminoglycans; and female hormone receptor activating factors such as estrogen and progesterone. Glycosaminoglycan derivatives include alkali metal salts such as sodium salts and potassium salts of glycosaminoglycan. When the pharmaceutical composition contains at least one of glycosaminoglycan and a derivative of the glycosaminoglycan, for example, the viscosity of the pharmaceutical composition increases, and the retention time of the pharmaceutical composition in the uterus may increase. When the pharmaceutical composition contains a female hormone receptor activating factor, proliferation and maturation of the endometrium may be further promoted. Examples of the liquid medium may include physiological saline and phosphate buffered saline (PBS).

The form of the pharmaceutical composition is not particularly limited as long as it is in the form of a preparation capable of being administered as a treatment of infertility. The form of the pharmaceutical composition in administration is preferably a liquid, more preferably a liquid capable of being gelled in the uterus.

As a method for administering the pharmaceutical composition, mention is made of, for example, a method in which an effective amount of the pharmaceutical composition is injected into a uterus of a subject, i.e. an intrauterine cavity. When the pharmaceutical composition is injected into the intrauterine cavity, and comes into contact with a surface of the endometrium, an implantation failure may be ameliorated by, for example, promoting angiogenesis in the endometrium, and growing the thinned endometrium unresponsive to estrogen. It is able to promote maturation of the endometrium, so that an implantation failure is ameliorated. Since it is able to proliferate the endometrium by injecting the pharmaceutical composition into the intrauterine cavity, the pharmaceutical composition does not damage tissues of a uterine muscle, and thus has extremely low invasiveness. Therefore, it is possible to easily and safely perform repeated administration. The pharmaceutical composition may be injected into the intrauterine cavity by, for example, a uterine catheter.

### Method for Producing Pharmaceutical Composition

A method for producing a pharmaceutical composition to be used for treatment of infertility includes treating an adipose tissue with a disaggregation agent to obtain a disaggregated tissue, concentrating reproductive cells from the disaggregated tissue by centrifugal separation treatment, and recovering the concentrated reproductive cells.

The adipose tissue to be used in the production method is collected from, for example, a subcutaneous tissue. The adipose tissue collected from the subcutaneous tissue may be washed with physiological saline, a buffered or non-buffered electrolyte solution. The adipose tissue is treated with a disaggregation agent to obtain a disaggregated tissue. Examples of the disaggregation agent include neutral protease, collagenase, trypsin, lipase, hyaluronidase, deoxyribonuclease and pepsin, and among them, collagenase is preferable. The disaggregation agent may be added to an adipose tissue together with other solution such as physiological saline. The treatment with the disaggregation agent can be performed by, for example, shaking the adipose tissue at or around a temperature of 37° C. for a time of from 20 minutes to 120 minutes.

From the disaggregated tissue obtained by treatment of the adipose tissue with the disaggregation agent, reproductive cells are concentrated by centrifugal separation treatment. It is preferable that prior to the centrifugal separation treatment, non-suspended matter components are removed from the disaggregated tissue to obtain a fraction including a suspended matter layer. The fraction including a suspended matter layer contains reproductive cells. The suspended matter layer containing reproductive cells may be subjected to washing treatment as necessary.

The reproductive cells concentrated by the centrifugal separation treatment are recovered by a conventional method to form a pharmaceutical composition. Details of a method for recovering reproductive cells from the above-described adipose tissue are described in, for example, Japanese Patent Laid-open Publication No. 2012-51923, Japanese Patent Laid-open Publication No. 2012-75439, International Publication No. WO 2015/042182 and International Publication No. WO 2006/127007. Reproductive cells may also be recovered from the adipose tissue using commercially available Celution System manufactured by Cytori Company.

### Method for Treatment of Infertility

A method for treatment of infertility is a method for treating infertility of a subject and the method includes administering an effective amount of a pharmaceutical composition containing adipose tissue derived reproductive cells (ADRCs) to an intrauterine cavity of a treatment subject. Adipose tissue derived reproductive cells contained in the pharmaceutical composition administered to the intrauterine cavity ameliorate an implantation failure to improve the implantation ratio. Adipose tissue derived reproductive cells contained in the pharmaceutical composition may be prepared at the time of use, or be frozen. The method for treatment of infertility may be a method for therapy of infertility.

Animals to be subjected to the method for treatment of infertility may be mammals, and the mammals include humans. An effective amount of ADRCs may be appropriately selected according to the animal species to be treated and the state of an individual. The number of ADRCs in an effective amount may be, for example, from 5×10⁵ to 5×10⁶. The pharmaceutical composition may be administered to the intrauterine cavity by a conventional method using a uterine catheter.

### Method for Amelioration of Implantation Failure

A method for amelioration of an implantation failure is a method for amelioration of an implantation failure in a subject, the method including administering an effective amount of a pharmaceutical composition containing adipose tissue derived reproductive cells (ADRCs) to an intrauterine cavity of a treatment subject to proliferate endometrial cells. Adipose tissue derived reproductive cells contained in the pharmaceutical composition may be prepared at the time of use, or be frozen.

Animals to be subjected to the method for amelioration of an implantation failure may be mammals, and the mammals include humans. An effective amount of ADRCs may be appropriately selected according to the animal species to be treated and the state of an individual. The number of ADRCs in an effective amount may be, for example, from 5×10⁵ to 5×10⁶ cells.

### EXAMPLES

Hereinafter, the present invention will be described more in detail by way of examples, but the present invention is not limited to these examples.

### Example 1

A mouse adipose tissue was collected subcutaneously from both limbs of a mouse (ICR, female, 5 weeks old). 10 ml of a 0.2% collagenase solution (GIBCO 17100-017) was added to 1 g to 2 g of the collected adipose tissue, and the adipose tissue was immersed, and finely cut by scissors. 20 ml of the 0.2% collagenase solution was added to the finely cut adipose tissue, and the mixture was shaken at 37° C. and 120 rpm for 1 hour. Thereafter, the mixture was filtered through a cell strainer (manufactured by FALCON Corporation, REF 352360), and the filtrate was subjected to a centrifugal treatment at 400 G for 5 minutes. An operation of removing the supernatant, suspending the resulting pellet in 10 ml of phosphate buffered saline (PBS), and subjecting the suspension to a centrifugal treatment at 400 G for 5 minutes was repeated three times to obtain a cell pellet. The resulting cell pellet was used as mouse ADRCs.

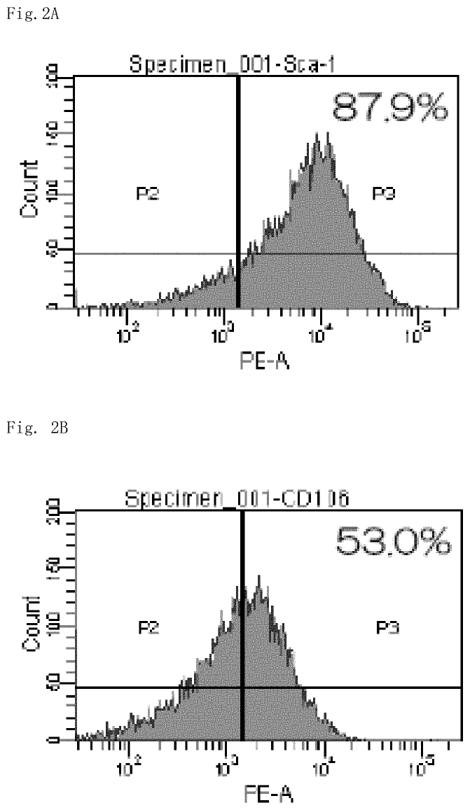

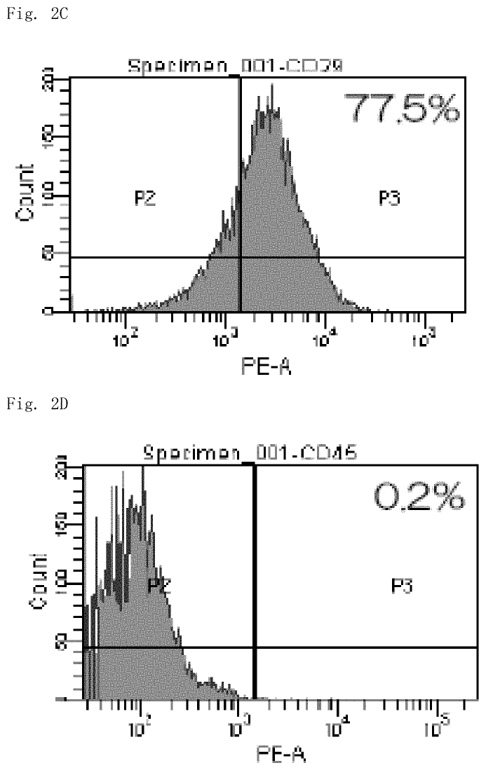

The resulting mouse ADRCs were stained using a multicolor flow cytometry kit (manufactured by R&D System Company, catalog number: FMC003), Sca-1, CD 105 and CD 29 as positive markers and a CD 45 antibody as a negative marker, and positive cells were detected by flow cytometry. The results are shown in FIG. 2. From FIG. 2, it is apparent that the ADRCs obtained as described above are positive for Sca-1, CD 105 and CD 29, and negative for CD 45.

### Example 2

For subjects who had agreed with a written informed consent, a human adipose tissue was collected from subcutaneous tissues of a waist-back part and a femur under general anesthesia. Cell pellets obtained by extracting reproductive cells from the collected human adipose tissue using Celution System manufactured by Cytori Company were used as human ADRCs.

### Preparation of Endometrial Thinning Model Mouse

A mouse (ICR, female, 5 weeks old) was incised at a back part, and an adipose tissue around a uterus was identified. The identified adipose tissue around the uterus was pulled to fix the uterus, and 95% ethanol was injected into the uterus to prepare an endometrial thinning model mouse.

### Test Example 1

### ADRCs Effectiveness Evaluation 1 Evaluation of Endometrial Proliferation Capability

The endometrial proliferation capability of mouse ADRCs was evaluated in the following manner using the endometrial thinning model mouse prepared as described above. A test drug was injected into the intrauterine cavity of the endometrial thinning model mouse 10 days after injection of 95% ethanol (after 2 cycles in the menstrual cycle). The condition of the endometrium was examined 7 days after injection of the test drug. As the test drug, a composition containing 1×10⁶ mouse ADRCs (30 μl of PBS) was injected for the test group, and physiological saline (30 μl) was injected for the control group. Nine model mice were used as the test group, and eight model mice were used as the control group. FIGS. 3, 4A, 4B, 5A and 5B show tissue hematoxylin/eosin (HE) staining views of the endometrium (magnification: 100).

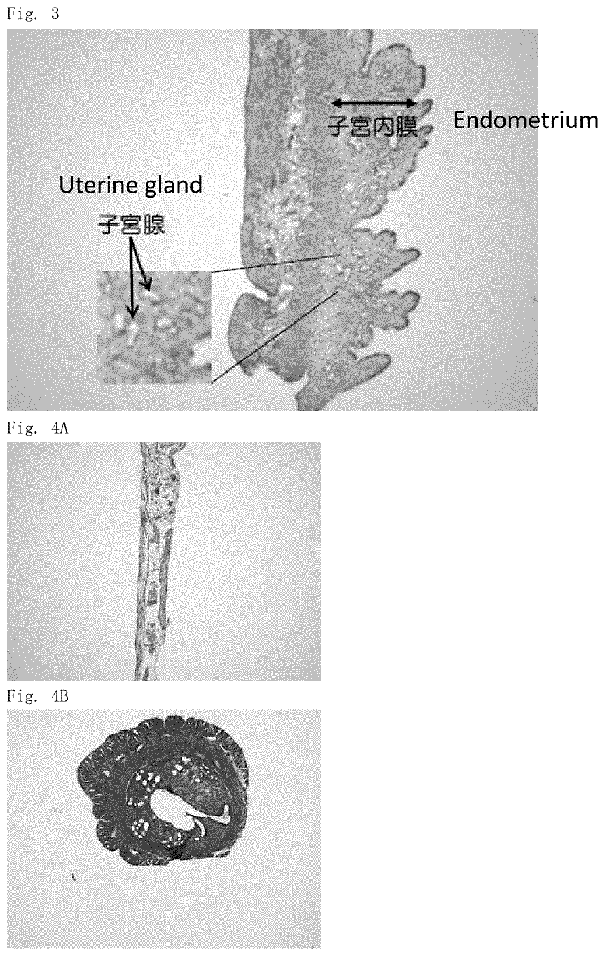

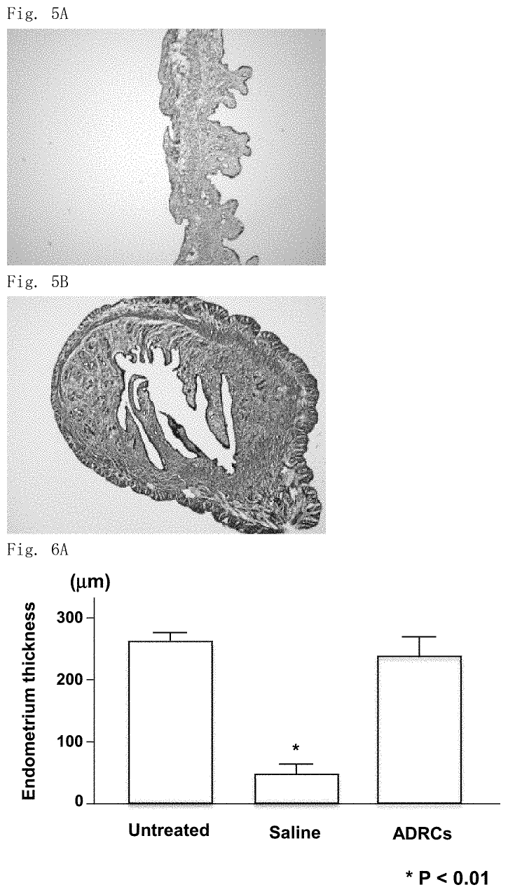

FIG. 3 is a tissue HE staining view showing the endometrium of a normal mouse. FIG. 4A shows a sagittal section of the uterus of the control group in which physiological saline is injected into the endometrial thinning model mouse, and FIG. 4B is a tissue HE staining view showing a traverse section of the uterus. FIG. 5A shows a sagittal section of the uterus of the test group in which mouse ADRCs are injected into the endometrial thinning model mouse, and FIG. 5B is a tissue HE staining view showing a traverse section of the uterus. In addition, FIG. 6A shows the thickness of the endometrium, and FIG. 6B shows the number of the uterine glands. The thickness of the endometrium was 54±5.2 μm (mean±SD, N=8) for the control group, and 268±28.6 μm (mean±SD, N=9) for the test group. Thus, the value of the thickness of the endometrium was significantly high for the test group in which ADRCs were injected (p<0.01). The number of uterine glands was 2.4±0.8 (mean±SD, N=8) for the control group, and 37±8.6 (mean±SD, N=9) for the test group. Thus, the value of the number of uterine glands was significantly high for the test group in which ADRCs were injected (p<0.01). For statistical examination, the Mann-Whitney's U test was used. Thus, it is apparent that by administering mouse ADRCs to the intrauterine cavity, the endometrium of the endometrial thinning model mouse is proliferated.

### Test Example 2

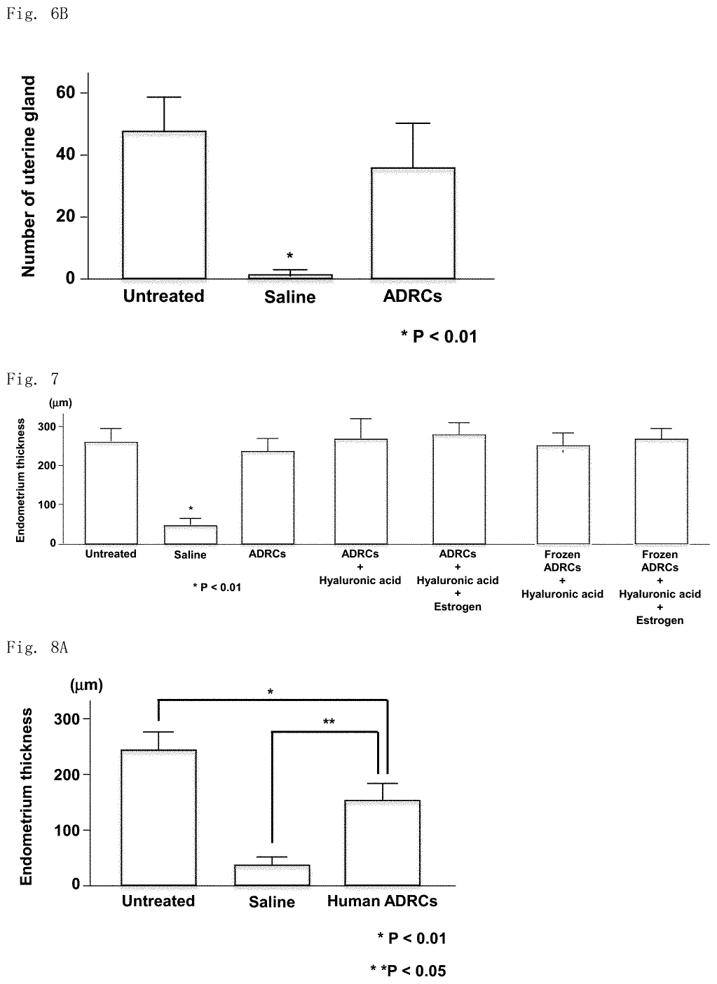

Using as test drugs a composition obtained by adding hyaluronic acid to mouse ADRCs, a composition obtained by adding hyaluronic acid and estrogen to mouse ADRCs, a composition obtained by adding hyaluronic acid to frozen mouse ADRCs, and a composition obtained by adding hyaluronic acid and estrogen to frozen mouse ADRCs, the endometrial proliferation capability was evaluated in the same manner as in Test Example 1. Here, PBS containing 1×10⁶ mouse ADRCs was used as mouse ADRCs, the addition amount of hyaluronic acid in the composition was 20 μL (0.2 mg), and the addition amount of estrogen was 3 μL (3 pg). The results of measuring the thickness of the endometrium are shown in FIG. 7.

From FIG. 7, it is apparent that addition of hyaluronic acid to ADRCs tends to improve the endometrial proliferation capability, and addition of estrogen further improves the endometrial proliferation capability. In addition, it is apparent that even after cryopreservation, ADRCs have an endometrial proliferation capability comparable to that of fresh ADRCs which is prepared at the time of use.

### Test Example 3

Endometrial proliferation capability in a mouse was evaluated in the same manner as in Test Example 1 except that in place of mouse ADRCs, human ADRCs were used as a test drug. FIG. 8A shows the thickness of the endometrium, and FIG. 8B shows the number of uterine glands as results.

From FIGS. 8A and 8B, it is apparent that even when human ADRCs are injected into the intrauterine cavity of the endometrial thinning model mouse, an endometrial proliferation capability is exhibited.

### Test Example 4

### ADRCs Effectiveness Evaluation 2 Evaluation of Angiogenesis Capability

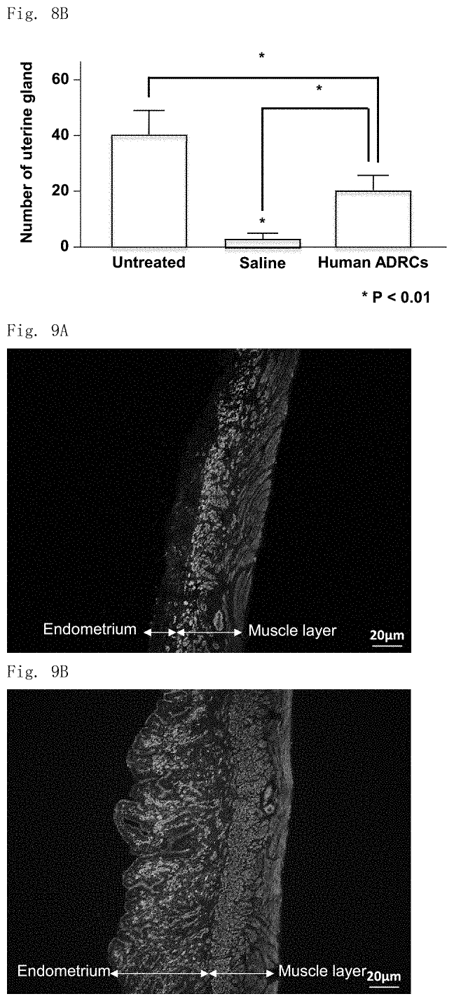

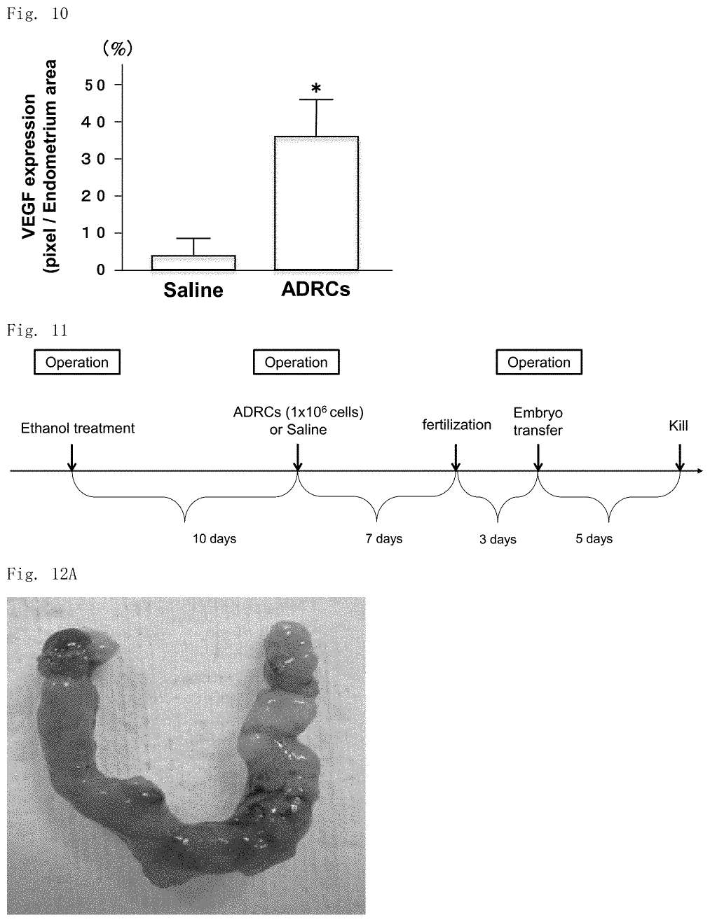

Except that the tissue was stained using an antibody for vascular endothelial cell growth factor (VEGF) fluorescent immunostaining (ab 46154 manufactured by Abcam plc.) instead of HE-staining the tissue, the same procedure as in Test Example 1 was carried out to evaluate the angiogenesis capability in the endometrium of mouse ADRCs using an endometrial thinning model mouse. FIGS. 9A, 9B and FIG. 10 show the results of VEGF fluorescence immunohistological staining of the endometrium.

FIG. 9A shows a sagittal section of the uterus of a control group in which physiological saline is injected into the intrauterine cavity of an endometrial thinning model mouse, and FIG. 9B is a fluorescent immunohistological staining view (magnification: 200) showing sagittal section of the uterine of a test group in which mouse ADRCs are injected into the intrauterine cavity of an endometrial thinning model mouse. In addition, FIG. 10 is a view in which the VEGF expression level is evaluated by the ratio (%) of a light-emission area in an endometrial region. The VEGF expression level in the endometrium was 3.4±2.5% (mean±SD, N=8) for the control group, and 34.8±9.5% (mean±SD, N=9) for the test group. Thus, the value of the VEGF expression was significantly high for the test group in which ADRCs were injected. In addition, it is apparent that VEGF is highly expressed particularly in an interstitial part of the endometrium.

### Test Example 5

### ADRCs Effectiveness Evaluation 3 Evaluation of Implantation Ratio

ADRCs effectiveness evaluation for the implantation ratio was performed in accordance with the experiment schedule shown in FIG. 11 using the endometrial thinning model mouse prepared as described above.

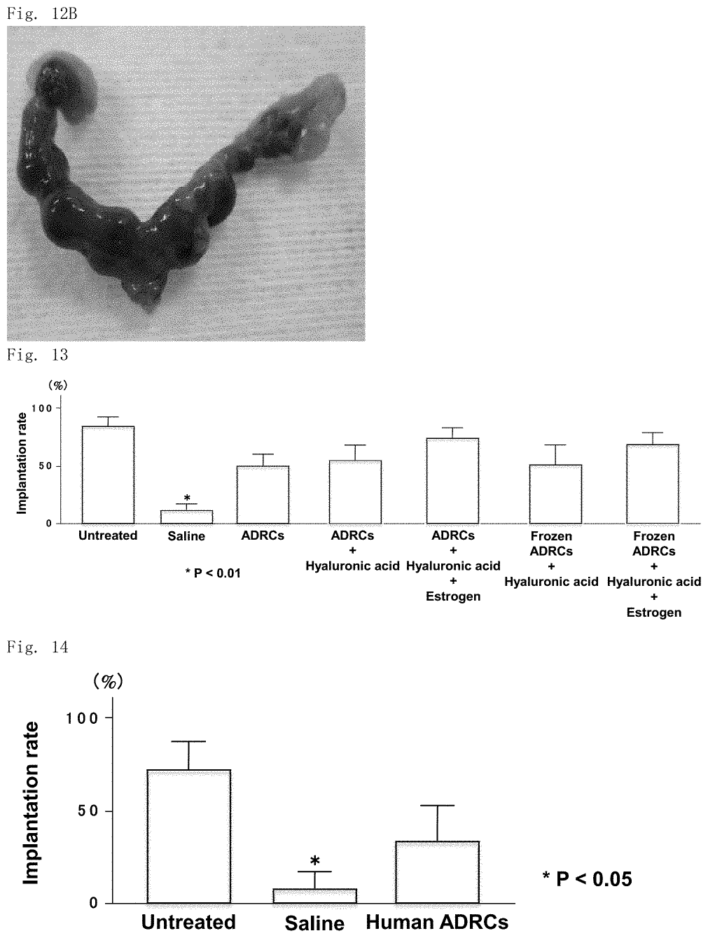

Specifically, the test drug was injected into the intrauterine cavity of the endometrial thinning model mouse ten days after injection of 95% ethanol. Thereafter, a mated embryo was implanted. Five days after implantation of the embryo, the mouse was sacrificed, and the implantation state of a fertilized egg was examined. As a test drug, a composition containing 1×10⁶ mouse ADRCs was used for a test group, and physiological saline was used for a control group. FIG. 12A shows an image of a uterus extracted from the control group, and FIG. 12B shows an image of a uterus extracted from the test group.

As shown in FIGS. 12A and 12B, implantation of the implanted embryo was not observed for the uterus of the control group in which physiological saline was injected into the intrauterine cavity of the endometrial thinning model mouse, and implantation of the implanted embryo was observed for the uterus of the test group in which ADRCs were injected.

### Test Example 6

Using as test drugs a composition obtained by adding hyaluronic acid to mouse ADRCs, a composition obtained by adding hyaluronic acid and estrogen to mouse ADRCs, a composition obtained by adding hyaluronic acid to frozen mouse ADRCs, and a composition obtained by adding hyaluronic acid and estrogen to frozen mouse ADRCs, the implantation state was examined to evaluate the implantation ratio in the same manner as in Test Example 5. Here, PBS containing 1×10⁶ mouse ADRCs was used as mouse ADRCs, the addition amount of hyaluronic acid in the composition was 20 μL (0.2 mg), and the addition amount of estrogen was 3 μL (3 pg). The results are shown in FIG. 13.

It is apparent that the test groups in which the composition containing mouse ADRCs was injected into the intrauterine cavity each had a higher implantation ratio as compared to the control group in which physiological saline was injected. In particular, in the composition with hyaluronic acid added to mouse ADRCs, the implantation ratio tended to be further improved, and in the composition with hyaluronic acid and estrogen added to mouse ADRCs, the implantation ratio tended to be still further improved.

### Test Example 7

The implantation state of an endometrial thinning model mouse was examined to evaluate the implantation ratio in the same manner as in Test Example 5 except that in place of mouse ADRCs, human ADRCs were used as a test drug. The results are shown in FIG. 14.

From FIG. 14, it is apparent that even when human ADRCs are injected into the intrauterine cavity of the endometrial thinning model mouse, the implantation ratio is improved.

### Test Example 8

### Verification of Quality of ADRCs

An adipose tissue was collected from a hip part of a miniature pig under general anesthesia, and ADRCs were collected using Celution System manufactured by Cytori Company. The abdomen of the miniature pig under general anesthesia was cut and opened to expose a uterus, the following ADRCs solution or control solution was injected into the uterine body part at two positions, and the abdomen was closed. The ADRCs solution was injected into the same individual as the individual from which adipose tissue was collected, and the control solution was injected into a different individual. Over the following 4 weeks, observation of the general condition of the miniature pig and body weight measurement (before injection, two weeks after injection, four weeks after injection) were performed. Blood tests were conducted before the injection and four weeks after the injection. Further, autopsy was performed four weeks after the injection.

2 ml of ADRCs solution: 0.7 ml of ADRCs (1×10⁶ cells) suspended in PBS+1.1 ml of hyaluronic acid*+0.2 ml of estrogen** (1)

2.0 ml of control solution: 0.7 ml of PBS+1.1 ml of hyaluronic acid*+0.2 ml of estrogen**

*Hyaluronic acid: sodium “Biochemistry” (Altz 25 mg/2.5 ml, SEIKAGAKU CORPORATION)**Estrogen: 17β-Estradiol (10 μg/ml in EtOH, Abcom (ab 120657))

### Results

As compared to control-administered individuals, individuals administered with ADRCs did not change their general condition over four weeks, and had no weight loss. For the blood test, there was no significant difference between the result before the injection and the result four weeks after the injection. There was no macroscopic abnormalities in the brain (cerebrum, cerebellum, medulla oblongata), the lung (including the bronchus), the heart, the liver (including the gall bladder), the spleen, the kidney, the adrenal and the implanted uterus, there was no significant difference in organ weight before the injection and four weeks after the injection, and there was no pathological abnormality.

The disclosure of Japanese Patent Laid-open Publication No. 2017-079148 (filing date: Apr. 12, 2017) is incorporated herein by reference in its entirety. All the documents, patent applications and technical standards described in this specification are incorporated herein by reference as if each document, patent application and technical standard were incorporated by reference, described specifically and individually.

What is claimed is:

1. A method for amelioration of an implantation failure, the method comprising administering adipose tissue derived reproductive cells or a pharmaceutical composition to an intrauterine cavity of a subject to proliferate endometrial cells,
wherein the pharmaceutical composition comprises adipose tissue derived reproductive cells including adipose stem cells, vascular endothelial cells and vascular pericytes.

2. The method according to claim 1, wherein the pharmaceutical composition further comprising at least one selected from the group consisting of glycosaminoglycan and a derivative of the glycosaminoglycan.

3. The method according to claim 2, wherein the glycosaminoglycan is hyaluronic acid.

4. The method according to claim 1, wherein the pharmaceutical composition further comprising an estrogen receptor activating factor.

5. The method according to claim 4, wherein the estrogen receptor activating factor is estrogen.

6. The method according to claim 1, wherein the pharmaceutical composition has an angiogenesis inducing action.

7. The method according to claim 1, wherein the pharmaceutical composition has an endometrial proliferation capability

8. The method according to claim 1, wherein the adipose tissue derived reproductive cells or adipose tissue derived reproductive cells contained in the pharmaceutical composition are prepared at time of use, or are frozen.

9. A method for treatment of infertility, the method comprising administering adipose tissue derived reproductive cells or a pharmaceutical composition to an intrauterine cavity of a subject,
wherein the pharmaceutical composition comprises adipose tissue derived reproductive cells including adipose stem cells, vascular endothelial cells and vascular pericytes.

10. The method according to claim 9, wherein the pharmaceutical composition further comprising at least one selected from the group consisting of glycosaminoglycan and a derivative of the glycosaminoglycan.

11. The method according to claim 10, wherein the glycosaminoglycan is hyaluronic acid.

12. The method according to claim 9, wherein the pharmaceutical composition further comprising an estrogen receptor activating factor.

13. The method according to claim 12, wherein the estrogen receptor activating factor is estrogen.

14. The method according to claim 9, wherein the pharmaceutical composition has an angiogenesis inducing action.

15. The method according to claim 9, wherein the pharmaceutical composition has an endometrial proliferation capability

16. The method according to claim 9, wherein the adipose tissue derived reproductive cells or adipose tissue derived reproductive cells contained in the pharmaceutical composition are prepared at time of use, or are frozen.
